(19) United States
(12) Patent Application Publication
(10) Pub. No.: US 2007/0238656 A1
(43) Pub. Date: Oct. 11, 2007

(54) Functionalized poly(ethylene glycol)

(75) Inventors: John W. Harder, Rochester, NY (US); Jeffrey W. Leon, Rochester, NY (US)

Correspondence Address: PAUL LEIPOLD;EASTMAN KODAK COMPANY, PATENT LEGAL STAFF, 343 STATE STREET, ROCHESTER, NY, 14650-2201, US

(73) Assignee: Eastman Kodak Company

(21) Appl. No.: 11/400,935

(22) Filed: Apr. 10, 2006

(51) Int. Cl. A61K 38/00

(52) U.S. Cl. 514/12

(57) ABSTRACT

The present invention relates to a bi-functional compound containing a linking material, and a particle comprising a linking material, and a linking material comprising a polyethylene glycol macromonomer backbone with a radical polymerizable group at one end of the macromonomer backbone and a different reactive chemical functionality at the other end of the macromonomer backbone, according to Formula I: 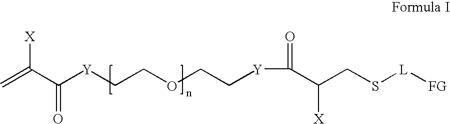
wherein X is CH₃, CN or H; Y is O, NR₁, or S; L is a linking group or spacer; FG is a functional group; n is greater than 4 and less than 1000; and wherein R₁ and R₂ are independently selected from substituted or unsubstituted alkyl, aryl, or heteroyl.

### CROSS REFERENCE TO RELATED APPLICATIONS

Reference is made to commonly assigned, co-pending U.S. patent applications: Ser. No. ______ by Leon et al. (Docket 92267) filed of even date herewith entitled “LOADED LATEX OPTICAL MOLECULAR IMAGING PROBES”, and Ser. No. ______ by Leon et al. (Docket 91032) filed of even date herewith entitled “NANOGEL-BASED CONTRAST AGENTS FOR OPTICAL MOLECULAR IMAGING”, the disclosures of which are incorporated herein by reference.

### FIELD OF THE INVENTION

The present invention relates to biocompatible polymeric linking materials.

### BACKGROUND OF THE INVENTION

Polyethylene glycol (“PEG” or “peg”) is one such chemical moiety which has been used in the preparation (“pegylation”) of therapeutic protein products (“pegylated proteins”). For example, pegylated adenosine deaminase is approved for treating severe combined immunodeficiency disease; pegylated superoxide dismutase has been used in clinical trials for treating head injury; pegylated alpha interferon has been tested in phase I clinical trials for treating hepatitis; pegylated glucocerebrosidase and pegylated hemoglobin are reported to have been in preclinical testing. For some proteins, the attachment of polyethylene glycol has been shown to protect against proteolysis, Sada et al., J. Fermentation Bioengineering 71:137-139 (1991). Methods for the attachment of certain polyethylene glycol moieties are available. See U.S. Pat. No. 4,179,337 (Davis et al.), and U.S. Pat. No. 4,002,531 (Royer).

For polyethylene glycol, a variety of means have been used to attach the polyethylene glycol molecules to the protein. Generally, polyethylene glycol molecules are connected to the protein via a reactive group found on the protein. Amino groups, such as those on lysine residues or at the N-terminus, are convenient for such attachment. For example, the Royer patent, above, states that reductive alkylation was used for attachment of polyethylene glycol molecules to an enzyme. European Patent Application 0 539 167, published Apr. 28, 1993, states that peptides and organic compounds with free amino group(s) are modified with an imidate derivative of PEG or related water-soluble organic polymers. U.S. Pat. No. 4,904,584 (Shaw) relates to the modification of lysine residues in proteins for the attachment of polyethylene glycol molecules via reactive amine groups.

Pegylation of protein molecules will generally result in a mixture of chemically modified protein molecules. As an illustration, protein molecules with five lysine residues and a free amino group at the N-terminus reacted in the above methods may result in a heterogeneous mixture, some having six polyethylene glycol moieties, some five, some four, some three, some two, some one, and some zero. Among the molecules with several, the polyethylene glycol moieties may not be attached at the same location on different molecules. The above methods typically require a linking moiety between the protein and the polyethylene glycol molecule. The procedure described by Delgado et al. in “Coupling of PEG to Protein by Activation with Tresyl Chloride, Applications In Immunoaffinity Cell Partitioning”, Separations Using Aqueous Phase Systems, Applications In Cell Biology and Biotechnology, Plenum Press, New York, N. Y. (1989), at pages 211-213, involves the use of tresyl chloride and results in no linking group between the polyethylene glycol and protein moieties. This method may be difficult to use to produce therapeutic products because the use of tresyl chloride may result in toxic by-products.

The conjugation of water-soluble polyalkylene oxides with therapeutic moieties such as proteins and polpeptides is known. See, for example, U.S. Pat. No. 4,179,337, the disclosure of which is hereby incorporated by reference. The '337 patent discloses that physiologically active polpeptides modified with PEG circulate for extended periods in vivo, have reduced immunogenicity and antigenicity.

To conjugate polyalkylene oxides, the hydroxyl end-groups of the polymer must first be converted into reactive functional groups. This process is frequently referred to as “activation” and the product is called an “activated polyalkylene oxide.”

For the most part, research has been directed to covalent attachment of polyalkylene oxides (PAO's) to epsilon amino groups of proteins, enzymes and polypeptides. Covalent attachment of polyalkylene oxides to lysine amino groups has been effected by linking groups such as succinoyl-N-hydroxysuccinimide ester, as disclosed by Abuchowski et al., Cancer Biochem Biophys., 7, 175-86 (1984), azlactones, aryl imidates and cyclic imide thiones. See U.S. Pat. Nos. 5,298,643, 5,321,095, and 5,349,001, for example. The contents of each of the foregoing patents are hereby incorporated by reference. PAO's have also been activated with hydrazine groups in order to couple the polymer to activated carbohydrate groups.

In addition to the foregoing, the conversion of terminal hydroxy groups of PAO's such as PEG to carboxylic acids has also been reported. PEG-acids are useful in at least two regards. First, carboxylic acid derivatives can be used directly to conjugate nucleophiles via available hydroxyl or amino moieties. Secondly, PAO carboxylic acids can be used as intermediates to form other types of activated polymers. For example, mPEG carboxylic acids can be converted to the succinimidyl ester derivative via N-hydroxysuccinimide and a condensing agent such as diisopropyl carbodiimide. Other activated PAO's can be prepared by reaction of the active ester with hydrazine to produce PAO-hydrazide derivatives.

The principal drawback in preparing carboxylic acid derivatives of polyalkylene oxides has been the difficulty in obtaining high yields of pure product. For example, Journal of Controlled Release, 10 (1989) 145-154 and Polymer Bulletin, 18, (1987), 487-493, describe the synthesis of mPEG acids by converting mPEG—OH to an ethyl ester followed by base catalyzed hydrolysis to form the carboxylic acid. Ostensibly, this classic approach should proceed without difficulty. In realty, however, this method at best provides m-PEG acids of about 90% purity, with the main product contaminant being the starting material, PEG—OH. In addition, the separation of the desired PEG acid from the starting PEG alcohol is very difficult. Standard laboratory methods such as fractional crystallization or column chromatography are not effective. Tedious column ion exchange or HPLC techniques provide purity of up to 95%, but these techniques are not suitable for large scale processes.

Preparation of a PEG-conjugated product, sometimes referred to as a pegylated product, using impure PEG carboxylic acids results in an mPEG—OH contaminated final product. For lower molecular weight peptides and organic conjugates, removal of the contaminant is very difficult due to the slight difference in molecular weight between the contaminant, mPEG—OH and the desired linking polymer conjugate. In addition, using lower purity polymer-carboxylic acid derivatives necessarily reduce the yield of the desired conjugates while adding to manufacturing costs due to the need to undertake tedious and expensive separation steps.

Kokai Patent Application No. HEI 9[1997]-255690 discloses a novel silane compound useful as a coupling agent, and inorganic microparticles being surface treated with the coupling agent. A novel silane compound is allowed to undergo the Michael addition reaction with the compound having two or more mercapto-group-containing silane and (meth) acryloyl functional groups in one molecule, and the inorganic microparticles are surface treated by the silane compound in hydrolysis. However if one desires to pegylate to biologically useful groups such as amino acids, peptides, antibodies, proteins, dyes, bioligands such as biotin or folic acid, or other useful organic compounds, then silane is a poor reactive group and is more useful to react with inorganic materials and surfaces.

US Patent Publication Number 2005/0176896 provides a method for preparing, in high purity and high yield, heterobifunctional derivatives of poly(ethylene glycol) or related polymers. A chromatographic purification step is not necessary in the method. In accordance with the method of the invention, an intermediate polymer having a formula of W-Poly—OH is provided bearing a removable group W at one terminus. The intermediate polymer W-Poly—OH is first altered by modifying the OH group to a first functional group X, followed by the removal of W to generate a second hydroxyl group. The latter hydroxyl group may then be further converted to a second functional group Y, thus providing the desired heterobifunctional derivative. However this material relies on converting one heterobifunctional derivative into another, since the starting material W-Poly—OH is a heterobifunctional polymer. It is more desirable to be able to convert a readily available homobifunctional polymer into a heterobifunctional polymer as in the present invention.

U.S. Pat. No. 5,756,593 relates to methods of preparing activated polyalkylene oxides. In particular, the invention relates to methods of preparing polyalkylene oxide carboxylic acids in high purity. The methods include reacting a polyalkylene oxide such as polyethylene glycol with a t-butyl haloacetate in the presence of a base followed by treatment with an acid such as trifloroacetic acid. The resultant polymer carboxylic acids are of sufficient purity so that expensive and time consuming purification steps required for pharmaceutical grade polymers are avoided. This method does not provide a way to make a heterobifunctional PEG in which the ends of the polyethylene glycol are substituted with different reactive groups such that the PEG group could be used to link to different materials

Article: Iyer et al., “Synthesis of orthogonal and functionalized oligoethylene glycols of defined lengths”, Tetrahedron Letters 45 (2004) pages 4285-4288. The described method is limited to small sized polyethyleneglycols because it relies on poor water soliblity of a symmetrical bis azide to achieve selectivity between the two end groups. 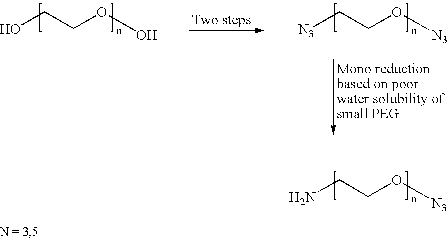
Medium to large sized heterobifunctional polyethyleneglycol groups with a free amine on one end and a methacrylate or methacrylamide could not easily be prepared by this method and are not disclosed as intermediates or products.

Article: Ehteshami et al., “Synthesis of monoprotected derivatives of homo-bifunctional molecules”, Reactive and Functional Polymers 35 (1997) pages 135-143 describes the synthesis of a symmetrical bis-amino-polyetlhyleneglycol that is reacted to put a blocking group on one end non-selectively followed by difficult chromotographic separation using costly materials.

Article: Riener et al., “Heterobifunctional crosslinkers for tethering single ligand molecules to scanning probes”, Analytica Chimica Acta 497 (2003) pages 101-114. A heterobifunctional polyethyleneglycol is prepared which cannot be used to prepare a latex because it has an amine on one end and carboxy group on the other group and the method requires difficult chromatography using costly materials.

### Problem to be Solved

There remains a need for an improved heterobifunctional polyethylene glycol that can be prepared without costly chromatography which contains functional groups which can be used to link contrast agents or therapeutic agents through a biocompatible PEG group, or form a biocompatible latex material which has reactive groups for the attachment of contrast agents and therapeutic agents or both.

### SUMMARY OF THE INVENTION

The present invention relates to a linking material comprising a polyethylene glycol macromonomer backbone with a radical polymerizable group at one end of the macromonomer backbone and a different reactive chemical functionality at the other end of the macromonomer backbone, according to Formula I: 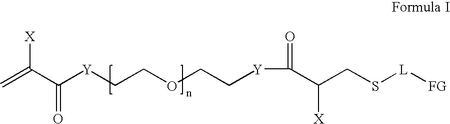
wherein X is CH₃, CN or H;
Y is O, NR₁, or S;
L is a linking group or spacer;
FG is a functional group;
n is greater than 4 and less than 1000; and
   wherein R₁ and R₂ are independently selected from substituted or unsubstituted alkyl, aryl, or heteroyl. The invention also relates to a bi-functional compound comprising a single linking material comprising a polyethylene glycol macromonomer backbone with a single radical polymerizable group at one end of the macromonomer backbone and a different reactive chemical functionality FG at the other end of the macromonomer backbone, according to Formula I: 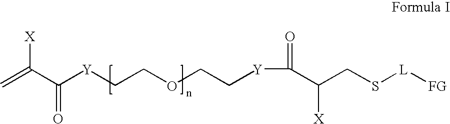
   wherein X is CH₃, CN or H;
Y is O, NR₁, or S;
L is a linking group or spacer;
FG is alkylated or acylated to a second functional compound;
n is greater than 4 and less than 1000; and
   wherein the single radical polymerizable group is reacted to a first functional compound;
   FG is NH₂, NHR₂ or COOH prior to alkylation or acylation to the second functional compound; and
   wherein R₁ and R₂ are independently selected from substituted or unsubstituted alkyl, aryl, or heteroyl. The invention also relates to a carrier particle comprising a particle having attached thereto a plurality of linking compounds comprising a polyethylene glycol macromonomer backbone with a single radical polymerizable group at one end of the macromonomer backbone, wherein the radical polymerizable group is reacted to the particle, and a different reactive chemical functionality FG at the other end of the macromonomer backbone, according to Formula I: 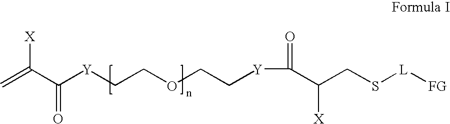
   wherein X is CH₃, CN or H;
Y is O, NR₁, or S;
L is a linking group or spacer;
FG is alkylated or acylated to a carried compound;
n is greater than 4 and less than 1000;
   wherein FG is NH₂, NHR₂ or COOH prior to the alkylation or acylation to the carried compound; and
   wherein R₁ and R₂ are independently selected from substituted or unsubstituted alkyl, aryl, or heteroyl.

### Advantageous Effect of the Invention

The present invention includes several advantages, not all of which are incorporated in a single embodiment. A linking group is provided that can connect two different biologically useful groups, and provides improved solubility in physiological environments, lower toxicity and immunogenicity. The specific end groups of the invention allow for two completely different processes to occur selectively, such as the formation of latex colloids and attachment of useful groups.

### DETAILED DESCRIPTION OF THE INVENTION

The present invention relates to a polyethylene oxide polymer backbone with specific end groups for use as a linking polymer in therapeutic and diagnostic materials for the analysis, detection and treatment of disorders in vitro and in vivo. Preferably, the linking polymer is a polyethylene glycol backbone chain with specific functional end groups at each end which allow the polyethylene glycol to act as a linking group between two materials through the two functional end groups.

The linking polymer is typically utilized in two ways. First, a single linking polymer may be used to attach one functional compound of interest to another, thereby producing a single compound with two different desired functions. Multiple linking polymers may also be attached to a single large particle or bead at one end and a compound of interest on the other, thereby producing a single carrier particle for a large payload of functional compound of interest.

For purpose of the present invention, the term:

“Pegylation” is the reaction by which a PEG-protein/peptide conjugate is obtained starting from the activated PEG and the corresponding protein/peptide. This may also apply to PEG-Therapeutic Agent, PEG-Dye, PEG-bioligand, PEG-(MRI Contrast Agent), PEG-(X-Ray Contrast Agent), PEG-Antibody, PEG-(Enzyme Inhibitor) PEG-(radioactive isotope), PEG-(quantum dot), PEG-oligosaccharide, PEG-polygosaccharide, PEG-hormome, PEG-dextran, PEG-oligonucleotide, PEG-carbohydrate, PEG-neurotransmitter, PEG-hapten, PEG-carotinoid.

The linking polymer may be used in both the acylation and alkylation approaches and is compatible with aqueous and organic solvent systems, so that there is more flexibility in reacting with useful groups and the desired products are more stable in an aqueous environment, such as a physiological environment. The linking polymer has a polyethylene glycol backbone structure from which depend at least two reactive groups, one at each end. The polyethylene glycol macromonomer backbone contains a radical polymerizeable group at one end. This group can be, but is not necessarily limited to a methacrylate, cyanoacrylate, acrylate, acrylamide, methacrylamide, styrenic, allyl, vinyl, maleimide, or maleate ester. The polyethylene glycol macromonomer backbone additionally contains a reactive chemical functionality at the other end which can serve as an attachment point for other chemical units, such as quenchers or antibodies. This chemically functional group may be, but is not limited to thiols, carboxylic acids, primary or secondary amines, vinylsulfonyls, aldehydes, epoxies, hydrazides, succinimidyl esters, maleimides, a-halo carbonyl moieties (such as iodoacetyls), isocyanates, isothiocyanates, and aziridines. Preferably, these functionalities will be carboxylic acids, primary amines, maleimides, vinylsulfonyls, or secondary amines. Most preferably, one of the reactive groups is an acrylate which is useful for forming nanogels and latexes and reacting with thiols through Michael addition, the other reactive groups is useful for conjugation to contrast agents, dyes, proteins, amino acids, peptides, antibodies, bioligands, therapeutic agents and enzyme inhibitors. Preferably, for therapeutic use of the end-product preparation, the linking polymer will be pharmaceutically acceptable. The polyethylene glycol macromonomer may have a molecular weight of between 300 and 10,000, preferably between 500 and 5000.

A particularly preferred water-soluble linking polymer for use herein is a polyethylene glycol derivative of Formula I. The polyethylene glycol (PEG) backbone of the linking polymer is a hydrophilic, biocompatible and non-toxic polymer of general formula H(OCH (₂)CH (₂)) (n)OH, wherein n>4. 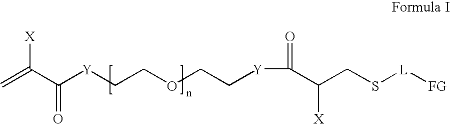
In Formula I:
X=CH3, CN or H, and, most preferably, X=CH3.
Y=O, NR₁, or S, and, most preferably, Y=O, NR₁.
   L is a linking group or spacer, preferably, substituted or unsubstituted alkyl, alkyloxy, aryl or heteroyl and may be unbranched, or branched to allow multiple functional groups (FG).
   FG is a functional group. FG may be NHCOR, NHSO₂R, NR2, SR, OR, NH₂, CO₂R, CONR2, SO₃H, SO2NR2, PO(OR)₃. Most preferably, FG is NH₂ or COOH. Functional group FG may preferably be halogen, haloacetamides, hydroxy, active esters, thiols, benzotriazole carbonates, p-nitrophenylcarbonates, isocyanates, and isothiocyanates, and most preferably is NH₂, NHR₂ or COOH. n is greater than 4 and less than 1000, preferably, n is between 6 and 500 or between 10 and 200. Most preferably, n=16.
   R₁ and R₂ are, independently, substituted or unsubstituted alkyl or aryl, or heteroyl, with preferred R₁ and R₂ groups chosen from alkyloxy, alkylhdydroxy, alkylamino, alkylcarbonamido, alkylcarbamoyl, alkylthioether, alkylthioester, aryloxy, arylamino, arylcarbonamido, arylcarbamoyl, arylnitro, arylthioester, arylthioether, arylcarboxyalkyl

The linking polymer may be used by attaching to biologically important materials, dyes and contrast agents for detection of disease and the study of metabolic activity, therapeutic agents for the treatment of disease, agents for making thickener agents, pharmaceuticals, and cosmetics. The preferred biologically important materials for attachment of the linking polymer include targeting agents, diagnostic agents, and therapeutic agents, which can be greatly improved in effectiveness when linked.

Targeting agents are compounds with useful groups that will identify and associate with a specific site, such as a disease site, such that the particle or conjugated material will be concentrated in this site for greater effect. Also of particular interest are PEG-antibodies. Antibodies, also known as immunoglobulins (Igs), are proteins that help identify foreign substances to the immune system, such as a bacteria or a virus or any substance bearing an antigen, and are useful for identification and association of specific biological targets. Bioligands are useful groups that will associate with receptor sites expressed in or on cells or with enzymes. Examples of bioligands are growth factors such as biotin and folic acid, specific proteins, and peptide sequences of amino acids or molecules which have strong binding ability to the active sites of enzymes or help the material penetrate or concentrate on or in cells of interest.

Diagnostic agents are materials which enhance the signal of detection when a material is scanned with light, sound, magnetic, electronic and radioactive sources of energy. Examples would be dyes such as UV, visible or infrared absorbing dyes especially fluorescent dyes such as indocarbocyanines and fluorescein, MIR contrast agents such as gadallinium and iron oxide complexes, and X-ray constrast agents such as a polyiodoaromatic compound.

Therapeutic agents are materials which effect enhance or inhibit cellular function, blood flow, or biodistribution, or bioabsorbtion. Examples would be pharmaceutical drugs for cancer, heart disease, genetic disorders, bacterial and virul infection and many other disorders.

Other useful materials to conjugate would be: PEG-peptide, PEG-protein, PEG-enzyme inhibitor PEG-oligosaccharide, PEG-polygosaccharide, PEG-hormome, PEG-dextran, PEG-oligonucleotide, PEG-carbohydrate, PEG-neurotransmitter, PEG-hapten, PEG-carotinoid.

The PEG could be functionalized with mixtures of these materials to improve effectiveness.

The following is a list of preferred linking polymers, but is not intended to an exhaustive and complete list of all linking polymers according to the present invention:

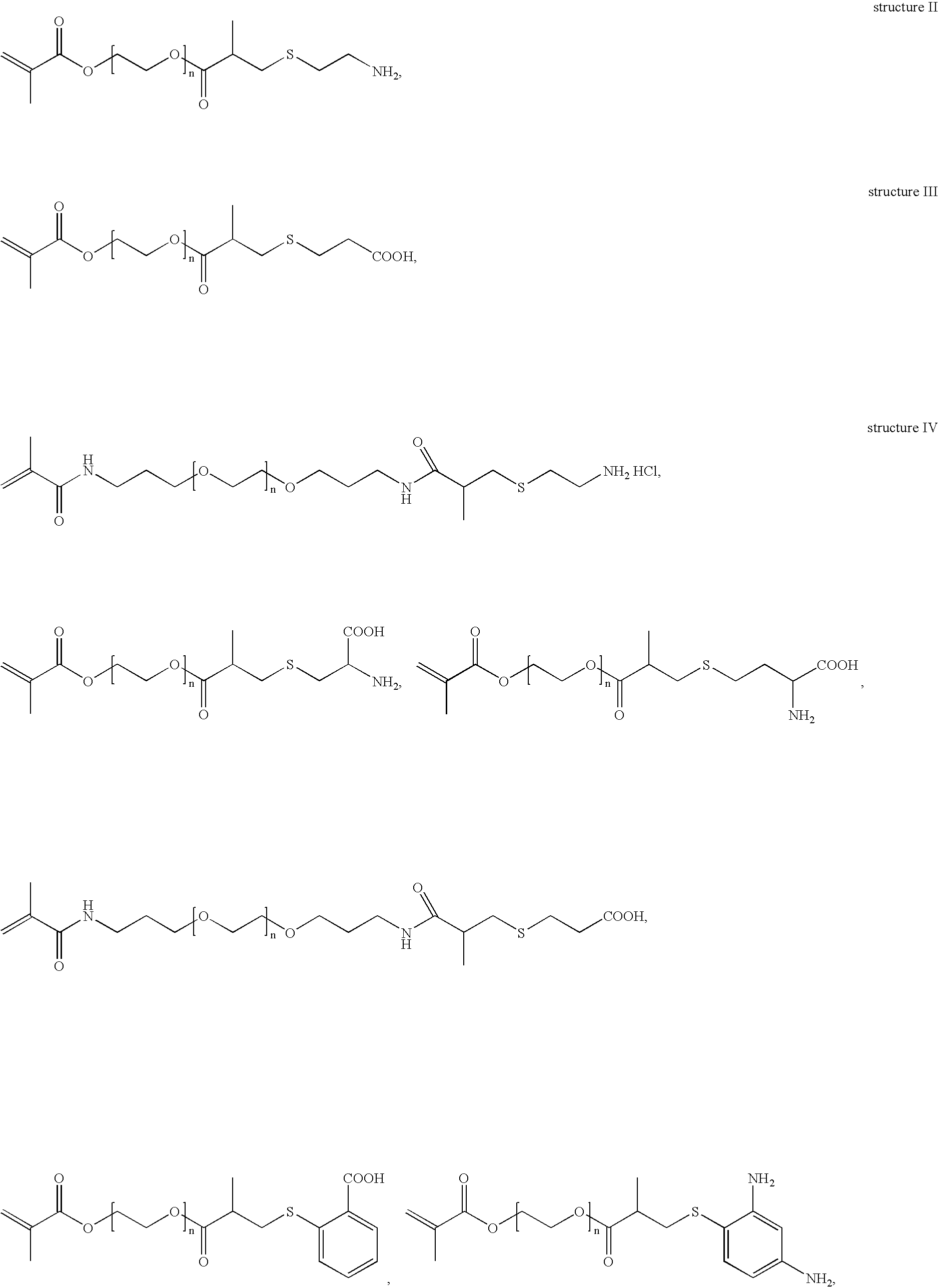

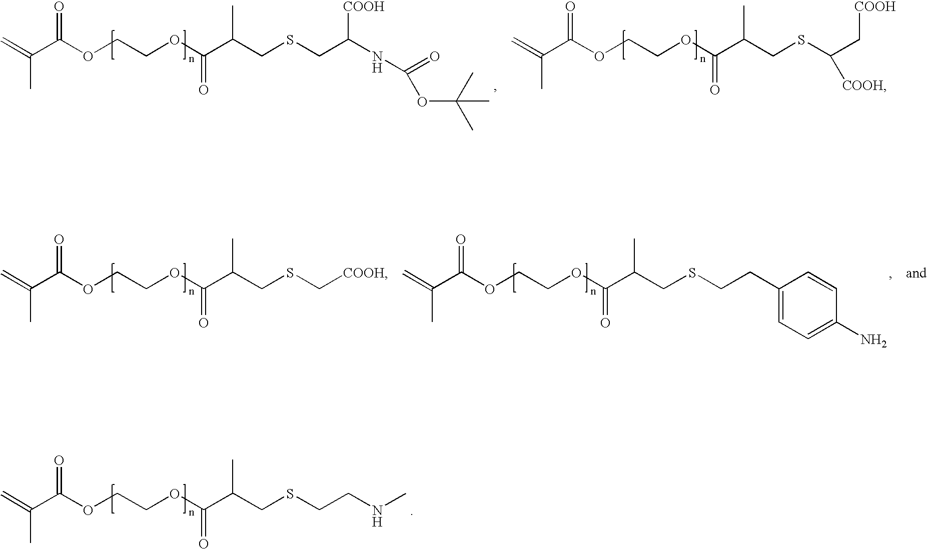

In one preferred method of use, multiple linking polymers are attached to a nanogel. For example, a first mixture of monomer(s) of interest, the linking polymer, and initiator is prepared in water. The first mixture was added to the second mixture of additional initiator and reacted, after which, additional initiator may be added to produce a nanogel composition. In another preferred method of use, multiple linking polymers are attached to a nanolatex. A mixture of monomers, linking polymer, initiator, surfactant, and buffer was prepared in water. The mixture is added to an aqueous solution of initiator, surfactant and buffer and reacted to produce a nanolatex particle according to the present invention.

In general, the derivatization may be performed under any suitable condition used to react a biologically active substance with an activated water soluble linking polymer molecule. In general, the optimal reaction conditions for the acylation reactions will be determined case-by-case based on known parameters and the desired result. For example, the larger the ratio of PEG: protein, the greater the percentage of polypegylated product. One may choose to prepare a mixture of linking polymer/polypeptide conjugate molecules by acylation and/or alkylation methods, and the advantage provided herein is that one may select the proportion of monopolymer/polypeptide conjugate to include in the mixture.

The following examples are provided to illustrate the invention.

### EXAMPLE A

### Hydroxyethyl Methacrylate-based Nanogel using Amine-terminated PEG Macromonomer.

A 500 ml 3-neck round bottomed flask was modified with Ace #15 glass threads at the bottom and a series of adapters allowing connection of 1/16 inch ID Teflon tubing. The flask (hereafter referred to as the “header” flask) was outfitted with a mechanical stirrer, rubber septum with syringe needle nitrogen inlet. The header flask was charged with hydroxyethyl methacrylate (3.91 g, 3.00×10⁻² mol), methylenebisacrylamide (0.12 g, 7.46×10⁻⁴ mol), the amine-terminated polyethylene glycol macromonomer of Example 1 (7.48 g, 7.57×10⁻³ mol), 2,2′-azobis(N,N′-dimethyleneisobutyramidine) dihydrochloride (0.12 g), and distilled water (72.11 g). A 1 L 3-neck round bottomed flask outfitted with a mechanical stirrer, reflux condensor, nitrogen inlet, and rubber septum(hereafter referred to as the “reactor”) was charged with (146.40 g), and 2,2′-azobis(N,N′-dimethyleneisobutyramidine) dihydrochloride (0.12 g). Both the header and reactor contents were stirred until homogeneous and were bubble degassed with nitrogen for 20 minutes. The reactor flask was placed in a thermostatted water bath at 50° C. and the header contents were added to the reactor over four hours using a model QG6 lab pump (Fluid Metering Inc. Syossett, N.Y.). When the addition was complete, a “chaser” of 2,2′-azobis(N,N′-dimethyleneisobutyramidine) dihydrochloride (0.04 g) was added and the reaction mixture was allowed to stir at 50° C. for 16 hours. The reaction mixture was then dialyzed for 48 hours using a 14K cutoff membrane in a bath with continual water replenishment. 252.0 g of a clear dispersion of 3.46% solids was obtained. The volume average diameter was found to be 25.8 nm with a coefficient of variation of 0.30 by quasi-elastic light scattering using a Nano ZS Model ZEN3600 (Malvern Instruments). Size exclusion chromatography in hexafluoro-2-propanol gave Mn=83,800, Mw=383,000, Mz=1,070,000

### EXAMPLE B

### Preparation of Nanolatex using Amine-terminated PEG Macromonomer.

This nanolatex was prepared using the same apparatus as described in Example A. The header contained methoxyethyl methacrylate (5.63 g), divinylbenzene (0.63 g, mixture of isomers, 80% pure with remainder being ethylstyrene isomers), poly(ethylene glycol) monomethyl ether methacrylate (6.25 g, Mₙ=1100), 2,2′-azobis(N,N′-dimethyleneisobutyramidine) dihydrochloride (0.06 g), cetylpyridinium chloride (0.31), sodium bicarbonate (0.06 g) and distilled water (78.38 g). The reactor contents were composed of distilled water (159.13 g), 2,2′-azobis(N,N′-dimethyleneisobutyramidine) dihydrochloride (0.06 g), sodium bicarbonate (0.06 g) and cetylpyridinium chloride (0.94 g). The reaction was carried out at 60C and the header was added over two hours. The reaction was allowed to proceed overnight. The latex was treated twice with 100 cc Dowex 88 ion exchange resin and dialyzed for 48 hours using a 14K cutoff membrane to afford to afford 312 g of a clear latex of 3.26% solids. The volume average diameter was found to be 20.89 nm with a coefficient of variation of 0.24 by quasi-elastic light scattering using a Nanotrac 150 Ultrafine Particle Analyzer (Microtrac Inc.).

### EXAMPLE 1

### Amine Preparation

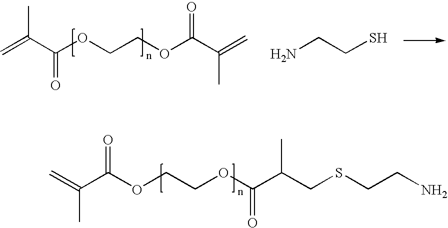

The polyethyleneglycol dimethacrylate (Aldrich, Mn 875) 335 g was mixed with 100 ml of methanol and treated with cysteamine (Aldrich, MW 77) 5.8g and diisopropylethylamine (Hunigs base) and stirred at RT for 2 days and concentrated. The residue was taken up in 1 L of ethyl acetate and extracted with aqueous 10% HCl. The aqueous layer was collected and made basic by the addition of 50% aqueous sodium hydroxide followed by extraction with ethyl acetate. The organic layer was dried over MgSO4, filtered and concentrated. The residue was taken up in anhydrous diethyl ether and treated with gaseous HCl and allowed to stand. The ether was decanted to leave a dark blue oil. This material was washed with fresh diethyl ether, which was decanted. The dark blue oil was concentrated by vacuum to give 37 g of the desired product as the hydrochloride salt.

¹H-NMR (300 MHZ,CDCl₃): D 1.18 (d, 3 H), 1.93 (bs, 3 H), 2.04 (bs, 2 H), 2.43-2.77 (bm, 7 H), 3.6-3.7 (vbs, —CH₂CH₂O—), 3.73 (bt, 2 H), 3.29 (bt, 2 H), 5.56 (bs, 1 H), 6.12 (bs, 1 H)

### EXAMPLE 2

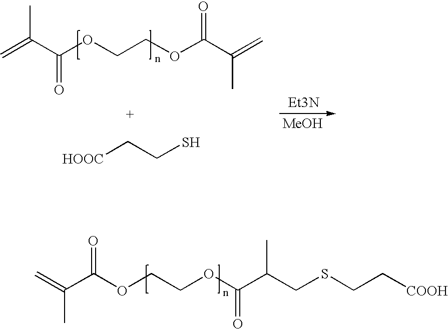

The polyethyleneglycol dimethacrylate (Aldrich, Mn 875) 300 g was mixed with 100 ml of methanol and treated with 3-mercaptopropionic acid (Aldrich, MW 106.14) 36.4 g and triethylamine (MW 101) 35 g and stirred at RT for 2 days and concentrated. The residue was taken up in 1 L of ethyl acetate and extracted with saturated aqueous sodium chloride. The organic layer was extracted twice with saturated aqueous sodium bicarbonate. The aqueous layers were combined and acidified with aqueous hydrogen chloride. The aqueous layer as then partitioned with ethyl acetate (twice). The combined organic layers were dried with magnesium sulfate, filtered and concentrated to give the desired product.

### EXAMPLE 3

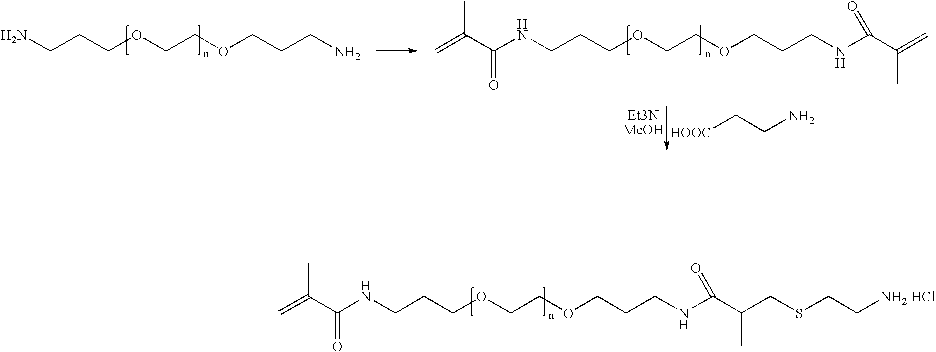

The bis-aminopropylpolyethyleneglycol (Mn 1500) 50 g was mixed with toluene (200 ml) and concentrated twice to remove water and dissolved again in toluene (200 ml) and treated with methacrylic anhydride (Mw 154) 11.2 g and stirred at room temperature for 24 hrs. The reaction was concentrated and taken up in toluene and concentrated again.

The polyethyleneglycol dimethacrylamide (Mn 1,910) 30 g was mixed with 100 ml of methanol and treated with cysteamine (Aldrich, MW 77) 0.4 g and triethylamine (MW 101) 3 g and stirred at RT for 2 days and concentrated. The residue was taken up in 200 ml of ethyl acetate and extracted with aqueous 10% HCl. The aqueous layer was collected and made basic by the addition of 50% aqueous sodium hydroxide followed by extraction with dichloromethane. The organic layer was dried over MgSO4, filtered and concentrated. The residue was taken up in anhydrous diethyl ether and treated with gaseous HCl and allowed to stand. The ether was decanted to leave a dark blue oil. This material was washed with fresh diethyl ether, which was decanted. The dark blue oil was concentrated by vacuum to give 37 g of the desired product as the hydrochloride salt.

### EXAMPLE 4

### Comparison of Functional Groups: Inventive Amine-functional to Silane Functional of Prior Art (pg. 4. Kokai Patent Application No. HEI 9[1997]-255690, Incorporated Herein by Reference) for Reactivity.

Compound 1 (inventive)or compound 2 (prior art comparison) were compared to determine the advantage of using an amine group vs. a trialkoxy silane group to attach organic compounds. The test compound (Compound 1 or Compound 2) was dissolved in ethylacetate and treated with the reactive group benzoic anhydride, N-phenylmethylcarbmoyl chloride, 4-methoxyphenyl isocyanate, or phenyl chloroformate with one equivalent of triethylamine. The reaction was evaluated by HPLC and mass spectra to determine if an adduct between the reactive group and the functionalized PEG compound had occurred.

| | |
|---|---|
| | Compound 1 (also, structure II) |
| 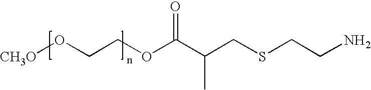 | |
| | Compound 2 |
| 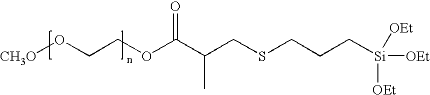 | |

| | | Product from |
|---|---|---|
| Reactive group | Product from Compound 1 | Compound 2 |
| Benzoic anhydride | 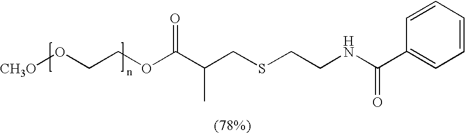 | No adduct (0%) |
| N-Phenylmethyl carbamoyl chloride | 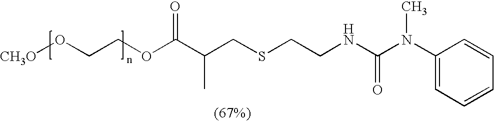 | No adduct (0%) |
| 4-Methoxyphenyl isocyanate | 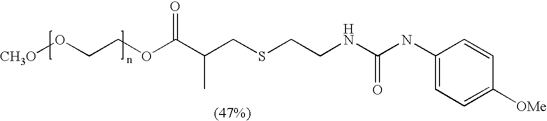 | Structure unknown (9%) |
| Phenyl chloroformate | 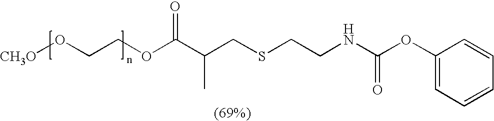 | No adduct (0%) |

This Example compares the usefulness of a linking compound with a functional end group which is silane (compound 2) against the same material with an amine-functional end (compound 1), in place of the silane functional group. Neither compound has the acrylate on it, as that part of the molecule would behave in a similar fashion. As can be seen from the Table above, the present material with a particular backbone bearing amine or carboxyl reactive groups is more capable of reacting with a variety of materials than the same backbone bearing other reactive groups known in the art.

The invention has been described in detail with particular reference to certain preferred embodiments thereof, but it will be understood that variations and modifications can be effected within the spirit and scope of the invention.

1. A linking material comprising a polyethylene glycol macromonomer backbone with a radical polymerizable group at one end of said macromonomer backbone and a different reactive chemical functionality at the other end of said macromonomer backbone, according to Formula I: 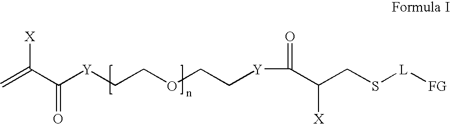
wherein X is CH₃, CN or H;
Y is O, NR₁, or S;
L is a linking group or spacer;
FG is a functional group excluding alkoxy silanes;
n is greater than 4 and less than 1000; and
wherein R₁ is selected from substituted or unsubstituted alkyl, aryl, or heteroyl.

2. The linking material of claim 1 wherein FG is selected from the group consisting of halogen, haloacetamides, hydroxy, active esters, thiols, benzotriazole carbonates, p-nitrophenylcarbonates, isocyanates, and isothiocyanates NH2, NHR₂ or COOH, wherein R₂ is independently selected from substituted or unsubstituted alkyl, aryl, or heteroyl.

3. The linking material of claim 1 wherein FG is NH2, NHR₂ or COOH, wherein R₂ is independently selected from substituted or unsubstituted alkyl, aryl, or heteroyl.

4. The linking material of claim 1 wherein FG is NH2 or COOH.

5. The linking material of claim 1 wherein X is CH3.

6. The linking material of claim 1 wherein Y is O or NR₁.

7. The linking material of claim 1 wherein L can be substituted or unsubstituted alkyl, alkyloxy, aryl or heteroyl.

8. The linking material of claim 1 wherein L is branched.

9. The linking material of claim 1 wherein n is between 10 and 200.

10. The linking material of claim 1 wherein n is between 6 and 500.

11. The linking material of claim 1 wherein n is 16.

12. The linking material of claim 1 wherein R₁ and R₂ are independently selected from the group consisting of alkyloxy, alkylhdydroxy, alkylamino, alkylcarbonamido, alkylcarbamoyl, alkylthioether, alkylthioester, aryloxy, arylamino, arylcarbonamido, arylcarbamoyl, arylnitro, arylthioester, arylthioether, and arylcarboxyalkyl.

13. The linking material of claim 1 wherein said polyethylene glycol macromonomer backbone has a molecular weight of from 300 to 10,000.

14. The linking material of claim 1 wherein said polyethylene glycol of Formula I is represented by the following structure II:

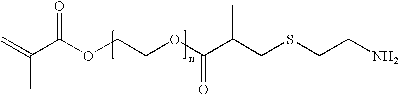

15. The linking material of claim 1 wherein said polyethylene glycol of Formula I is represented by the following structure III:

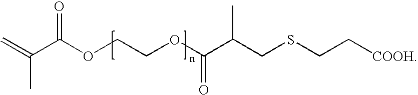

16. The linking material of claim 1 wherein said polyethylene glycol of Formula I is represented by the following structure IV:

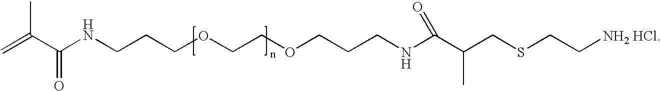

17. The linking material of claim 1 wherein said radical polymerizable group at one end of said macromonomer backbone is capable of Michael addition.

18. The linking material of claim 1 wherein FG is capable of alkylation or acylation.

19. The linking material of claim 1 wherein said linking material is utilized in an aqueous physiological environment.

20. A bi-functional compound comprising a single linking material comprising a polyethylene glycol macromonomer backbone with a single radical polymerizable group at one end of said macromonomer backbone and a different reactive chemical functionality FG at the other end of said macromonomer backbone, according to Formula I: 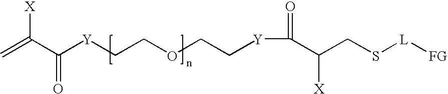
wherein X is CH₃, CN or H;
Y is O, NR₁, or S;
L is a linking group or spacer;
FG is alkylated or acylated to a second functional compound;
n is greater than 4 and less than 1000; and
wherein said single radical polymerizable group is reacted to a first functional compound;
FG is NH₂, NHR₂ or COOH prior to alkylation or acylation to said second functional compound; and
wherein R₁ and R₂ are independently selected from substituted or unsubstituted alkyl, aryl, or heteroyl.

21. The bi-functional compound of claim 20 wherein said first functional compound is a nanogel, a latex or a compound having a thiol group.

22. The bi-functional compound of claim 20 wherein said second functional compound is at least one member selected from the groups consisting of contrast agents, dyes, proteins, amino acids, peptides, antibodies, bioligands, targeting agents, diagnostic agents, therapeutic agents and enzyme inhibitors.

23. A carrier particle comprising a particle having attached thereto a plurality of linking compounds comprising a polyethylene glycol macromonomer backbone with a single radical polymerizable group at one end of said macromonomer backbone, wherein said radical polymerizable group is reacted to said particle, and a different reactive chemical functionality FG at the other end of said macromonomer backbone, according to Formula I: 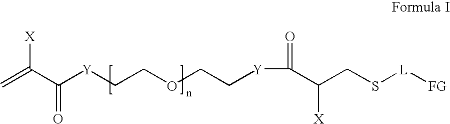
wherein X is CH₃, CN or H;
Y is O, NR₁, or S;
L is a linking group or spacer;
FG is alkylated or acylated to a carried compound;
n is greater than 4 and less than 1000;
wherein FG is NH₂, NHR₂ or COOH prior to said alkylation or acylation to said carried compound; and
wherein R₁ and R₂ are independently selected from substituted or unsubstituted alkyl, aryl, or heteroyl.

24. The carrier particle of claim 23 wherein said particle is a nanogel, a latex, or a particle with thiol groups for reacting through Michael addition.

25. The carrier particle of claim 23 wherein said carried compound is at least one member selected from the groups consisting of contrast agents, dyes, proteins, amino acids, peptides, antibodies, bioligands, targeting agents, diagnostic agents, therapeutic agents and enzyme inhibitors. t the
